(19) United States
(12) Patent Application Publication
(10) Pub. No.: US 2022/0225039 A1
(43) Pub. Date: Jul. 14, 2022

(54) SELF-FITTING HEARING AID HAVING BUILT-IN PURE TONE SIGNAL GENERATOR

(71) Applicant: ZHENGDONG HUANG, YANCHENG (CN)

(72) Inventor: ZHENGDONG HUANG, YANCHENG (CN)

(21) Appl. No.: 17/610,145

(22) PCT Filed: Jan. 21, 2020
(86) PCT No.: PCT/CN2020/073645

§ 371 Date: Nov. 9, 2021

(30) Foreign Application Priority Data
May 27, 2019 (CN) 201910444098.2

(51) Int. Cl. H04R 25/00

(52) U.S. Cl. CPC H04R 25/70; H04R 25/558; A61B 5/123; H04R 2225/55; H04R 25/505

(57) ABSTRACT

A self-fitting hearing aid having built-in pure tone signal generator is disclosed, comprising a hearing aid and external software. self-fitting is performed to experience a compensation effect through cooperating the compensation form provided by an digital signal processing system with corresponding App fitting software on a computer or mobile phone terminal. Hearing-impaired persons can directly perform self-hearing tests, and the system compiles corresponding parameters, such as frequency, decibel, inflection point and compression ratio according to data of the self-hearing tests, without external devices for various tests. Differences in parameters of the external test devices are avoided through the internal digital signal processing system and the built-in hearing test module in the hearing aid. A speaker inside the same hearing aid is used to the test-fitting and the hearing aid in the disclosed, which simplifies fitting procedures and improves the accuracy of the hearing test and fitting.

### TECHNICAL FIELD

The present disclosure relates to the technical field of hearing aids, in particular to a self-fitting hearing aid having built-in pure tone signal generator.

### BACKGROUND

Hearing aids are mainly composed of three main parts: a chip, a housing, and a sound-guiding tube. According to shapes and structures, the hearing aids are divided into a completely-in-canal (CIC), an in-the-canal (ITC), a middle-in-the-ear (MITE), a completely in-the-ear (ITE), a behind-the-ear (BTE), and a cassette-type (PCT); and the hearing aids are divided into 01 to 32 passages according to the passages. The hearing aids are suitable for hearing impaired persons whose residual hearing ranges are between 30 dB and 110 dB HL.

At present, an existing hearing aid is a simple device that only amplifies a sound and processes the sound. If the hearing aid is to be debugged and programmed, special external devices are required, such as audiometer (a testing device for testing the degree of hearing loss, a pure tone signal generator and a control part), programmer, fitting software on a computer, connecting wire, etc., thus lots of devices are required, and professional fitting technicians are needed, which will cause great inconveniences to the hearing impaired persons. Moreover, the fitting effects will have different degrees of deviations due to the differences in the parameters of the test devices and the hearing aid, which easily causes that the hearing aid fitting cannot be matched with the hearing impaired person completely and accurately, thereby affecting the accuracy of testing and fitting of the hearing aid. Based on the above, a self-fitting hearing aid having built-in pure tone signal generator is provided hereon.

### SUMMARY

The present disclosure provides a self-fitting hearing aid having built-in pure tone signal generator, which has the functions of improving the accuracy of hearing test and fitting, and simplifying the fitting procedures, thus to solve the problems mentioned in the prior art and to achieve self-testing.

The above objects are achieved by the following technical solutions according to present disclosure.

A self-fitting hearing aid having built-in pure tone signal generator comprises two parts: a hearing aid and external software.

The hearing aid comprises a audio amplification processing module and a pure tone signal generator module, the audio amplification processing module is a digital signal processing system, and the pure tone signal generator module is built into a digital processing chip. The external software is composed of hearing test software, a pure tone signal generator, and programming software, wherein the hearing test and the programming are intelligent. The self-fitting hearing aid having built-in hearing test is used for target hearing-impaired patients; by using the built-in pure tone signal generator in the hearing aid and the corresponding App fitting software on the external computer or mobile phone, the hearing-impaired patients can perform professional fitting services on another device with the transmitted data values, without fitting technicians. The hearing-impaired patients can directly wear the hearing aids. Self-fitting can be realized anywhere (when the network is available) by cooperating the compensation form provided in the digital signal processing system with the corresponding fitting software on the computer or mobile phone, and the compensation effects can be experienced through the listening experience module.

Alternatively, the external software is arranged in a corresponding App inside a computer or mobile phone terminal or a special fitting box, and an interface of the external software further comprises main buttons such as a retest button, a save button, and a clicking button after hearing.

Alternatively, the external software is connected to the self-fitting hearing aid having built-in pure tone signal generator in a wired or wireless manner, the pure tone signal generator within the external software is connected to the hearing aid without built-in hearing test in a wired or wireless manner, and the both manners can realize self-fitting.

Alternatively, a hearing loss compensation chart is represented by a coordinate graph, wherein an X axis is frequency, and a Y axis is composed of decibels and etc.

Alternatively, the software in the self-fitting system is provided with a pure tone signal generator that is used for connecting to a hearing aid without built-in pure tone signal generator for self-fitting. The pure tone signal generator in the system sends test signals to a user through a mobile phone headset or the hearing aid, thus to obtain the hearing loss compensation chart. The self-fitting system will automatically compile parameters settings, such as frequency, decibel, inflection point, compression ratio, etc., according to data of the hearing loss compensation chart.

An embodiment of the present disclosure provides a self-fitting hearing aid having built-in pure tone signal generator, which comprises two parts: a hearing aid and external software.

The hearing aid comprises a audio amplification processing module and a pure tone signal generator module, and the pure tone signal generator module is built into a digital processing chip.

The external software comprises hearing test software, a pure tone signal generator, and a programming module.

The self-fitting hearing aid is used as: aiming at target hearing-impaired patients, a built-in pure tone signal generator within the hearing aid is connected and matched with an external fitting module to perform self-fitting, and to experience compensation effects through a listening experience module.

Preferably, the external software is arranged in an App of a smart device or in a matched fitting box.

An interface of the external software further comprises at least a retest button, a save button, and a clicking button after hearing button (it can be a physical button or a virtual button with touch function).

Preferably, the external software is connected to the self-fitting hearing aid having built-in pure tone signal generator in a wired or wireless manner for realizing self-fitting.

Preferably, the pure tone signal generator within the external software is connected to the hearing aid without built-in hearing test in a wired or wireless manner for realizing self-fitting.

Preferably, the external software is provided with a pure tone signal generator that is used for connecting to the hearing aid without built-in pure tone signal generator for performing self-fitting; the provided pure tone signal generator sends test signals to a user through a mobile phone headset or the hearing aid, thereby forming hearing loss compensation parameters.

Preferably, the hearing loss compensation parameters comprise one or a combination of frequency, decibel, inflection point, and compression ratio.

The present disclosure provides a self-fitting hearing aid having built-in pure tone generator, which has the following beneficial effects:

1. According to the self-fitting hearing aid having built-in pure tone generator, self-fitting is performed to experience a compensation effect through the compensation form provided by an digital signal processing system within the hearing aid and cooperating with corresponding App fitting software on a computer or mobile phone terminal. A hearing-impaired patient can directly perform a self-hearing test, and the system will compile corresponding parameters, such as frequency, decibel, inflection point and compression ratio, according to data of the self-testing, without external devices for various tests. The internal digital signal processing system and a built-in hearing test module are provided in the hearing aid to avoid the differences in parameters of the external test devices. The test and fitting and hearing aid of the present disclosure adopts a speaker inside the same hearing aid, which can simplify the fitting procedures and improve the accuracy of the hearing test and fitting.

2. According to the self-fitting hearing aid having a built-in pure tone generator, the self-fitting can be realized anywhere (when the network is available) by cooperating the digital signal processing system with the corresponding App in the computer or mobile phone, without external devices and professional fitting specialists, which can greatly facilitate hearing-impaired persons and elderly users, simplify the fitting procedures and realize the self-fitting.

### DRAWINGS

FIG. 1 is a hearing analysis compensation chart according to an embodiment of the present disclosure.

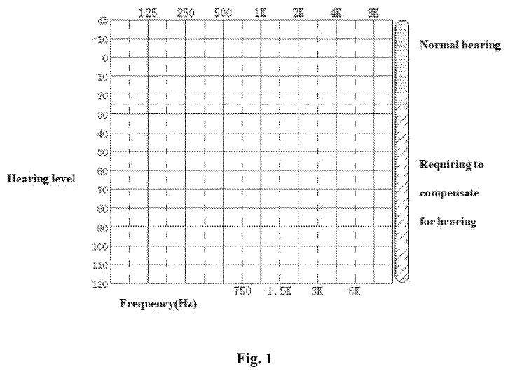

### DETAILED DESCRIPTION OF PREFERRED EMBODIMENTS

Embodiment 1 provides a self-fitting hearing aid having built-in pure tone signal generator, which comprises two parts: a hearing aid and external software.

The hearing aid comprises a audio amplification processing module and a pure tone signal generator module, wherein the audio amplification processing module is a digital signal processing system, and a hearing test module is built into a digital processing chip. The external software is composes of hearing test software, a pure tone signal generator and programming software. Testing and programming are intelligent. With the digital signal processing system, the self-fitting can be realized anywhere (when the network is available) through a corresponding App in a computer or mobile phone, without external devices and professional fitting specialists, which can greatly facilitate hearing-impaired persons and elderly users, simplify the fitting procedures and realize self-fitting.

In the digital signal processing system, a relevant hearing analysis compensation chart can be obtained after the test to the target hearing impaired person. The hearing impaired person can transmit sound waves to another device for fitting and other operations without professional fitting specialists. The hearing impaired patient can wear the hearing aids directly, the self-fitting can be realized anywhere (when the network is available) by cooperating the compensation form provided in the digital signal processing system with the corresponding fitting App software on the computer or mobile phone terminal, and the hearing impaired patients can experience the compensation effect through the hearing experience module, so as to avoid differences in parameters of the external test devices. The speaker inside the same hearing aid is used in the testing, fitting, and hearing aid according to the present disclosure, thus the present disclosure can simplify the fitting procedures and improve the accuracy of the hearing test and fitting.

The external software is arranged in a corresponding App in the computer or mobile phone terminal or in a special fitting box, and an interface of the external software further comprises main buttons such as a retest button, a save button, and a clicking button after hearing. When in use, user can click the retest button to re-fit according to his/her own fitting situations or when he/she feels that the fitting effects are not ideal.

The hearing aid is connected through a wired or wireless manner, and then the pure tone signal generator in the hearing aid sends out pure tone signals with different frequencies and different gains through the hearing test software, and finally a speakers in the hearing aid generates sound, and accordingly, the test software sends out frequency commands of 125, 250, 500, 750, 1K, 1.5K, 2K, 3K, 4K, 6K, 8K (Hz) respectively. The frequency points are settable, and a volume of each frequency point is started from 20 dB (settable), which allows the user to confirm whether he/she can hear the pure tone signals in the hearing aid. If the user cannot hear the pure tone signals, the system will automatically increase the volume by 3-5 dB (settable) after 3-10 seconds (settable). If the user can hear the pure tone signals, a confirmation button can be clicked, thus the system will automatically record a hearing threshold value of a certain frequency point (hearing threshold coordinate points of the frequency and decibel). If the user still cannot hear the pure tone signals, the system will continue to automatically increase the volume by 3-5 dB until the user can hear the pure tone signals.

By analogy, the frequency commands of 125, 250, 500, 750, 1K, 1.5K, 2K, 3K, 4K, 6K, 8K (Hz) sent out by the test software reach the user's audible range. After hearing the pure tone signals, the user can press the confirmation button on the software interface, thus the system will automatically record the hearing threshold of the frequency point.

After the specified frequency points have been tested, the system will automatically generate the user's hearing loss parameter charts (the system is automatically connected to each hearing threshold to form the hearing loss compensation chart). Based on the user's hearing loss parameter charts, the software programming system will automatically assemble the fitting formulas in the programming system and calculate a set of related parameters suitable for the user, and then automatically adapt program settings in the voice processing system of the hearing aid in a wired or wireless manner. The external software is connected to the self-fitting hearing aid having built-in pure tone signal generator in a wired or wireless manner.

The hearing loss compensation chart is represented by a coordinate graph, wherein an X axis is frequency, and a Y axis is composed of decibel, etc. The present disclosure is suitable for all hearing-impaired persons and greatly facilitates the users to perform self-fittings for the hearing aids by themselves. Moreover, due to changes in the degree of the hearing loss, self-fitting can be performed from time to time, or self-fitting can be performed again every six months.

The pure tone signal generator is provided within the self-fitting system software and used for self-fitting for the hearing aid without built-in pure tone signal generator. The pure tone signal generator within the self-fitting system sends test signals to the user through a mobile phone headset or the hearing aid, thus to obtain the hearing loss compensation charts. The self-fitting system automatically compiles parameters such as frequency, decibel, inflection point, compression ratio, etc., according to the data of the hearing loss compensation charts.

Embodiment 2, which is different from Embodiment 1 in that: the built-in hearing test module can be designed as an independent unit in the digital voice processing chip, or can be designed as an independent unit outside the chip but inside the hearing aid. By having the built-in hearing test module designed inside the hearing aid, it helps to reduce the occupied space. When the built-in hearing test module is designed as an independent unit and located in the hearing aid at the same time, the internal speaker of the same hearing aid is used to perform fitting, which can simplify the fitting procedures, avoid that the frequency response curve mismatch occurs when the hearing aid collects relevant data, and improve the accuracy of the hearing test and fitting.

Embodiment 3, which is different from Embodiment 1 and Embodiment 2 in that: the pure tone signal generator module can also be built in a form of pure software (algorithm) and built into the corresponding App software on the computer or mobile phone terminal that is matched with the self-fitting hearing aid having built-in pure tone signal generator, which is convenient for the users to process and store the data of the hearing test module, monitor the hearings for the hearing impaired persons, and record the data in the debugging software conveniently and quickly. The patient's hearing audiogram can be obtained quickly and accurately through a built-in audiometry, and the hearing aid is connected in a wired or wireless manner, and meanwhile, when the hearing-impaired person feels that there may be large errors in his/her hearing audiogram, self-testing can be performed to supplement and verify, or to program the data through the corresponding App software on the computer or mobile phone terminal, thereby realizing self-fitting.

Operation steps: the user connects the hearing aid in a wired or wireless manner through the fitting software of the corresponding App on the computer or mobile phone when there is a network. Firstly, the pure tone signal generator in the hearing aid emits pure tone signals of different frequencies and different gains through the hearing test software, and a speaker in the hearing aid emits sounds. Then, the test software respectively sends out corresponding sound hertz frequency commands, and the users can hear the pure tone signals in the hearing aid respectively. If the users cannot hear the pure tone signals, the system will automatically increase the volume within the corresponding time until the user can hear the pure tone signals. After hearing the pure tone signals, the users can press a confirmation button on the software interface, and the system will automatically record the hearing threshold of the frequency point. Finally, the relevant parameters suitable for the users are calculated through the software programming system, and then the program settings in the voice processing system of the hearing aid are automatically rewritten in a wired or wireless manner. If the users feel that the hearing effects are not good, the retest button can be press for retesting. After the adaptation is completed, the users can press a clicking button after hearing on the software interface. The next time the user use it, just turn on the switch. If the hearing loss degree has changed, a retesting is required again.

It should be noted that, in the present disclosure, the relational terms such as first and second are only used to distinguish one from another entity or operation without necessarily requiring or implying any actual such relationship or order between such entities or operations. Besides, the terms “include”, “comprise” or any other variants thereof are intended to cover non-exclusive inclusion, so that a process, method, article or device of including a series of elements not only includes those elements, but also includes other elements that are not explicitly listed, or also includes the elements inherent to the process, method, article or device.

What is claimed is:

1. A self-fitting hearing aid having built-in pure tone signal generator, comprising two parts: a hearing aid and external software,
wherein the hearing aid comprises a audio amplification processing module and a pure tone signal generator module, and the pure tone signal generator module is built into a digital processing chip,
the external software comprises hearing test software, a pure tone signal generator, and a programming module,
the self-fitting hearing aid is used as: aiming at target hearing-impaired patients, a built-in pure tone signal generator within the hearing aid is connected and matched with an external fitting module to perform self-fitting, and to experience compensation effects through a hearing experience module.

2. The self-fitting hearing aid having built-in pure tone signal generator according to claim 1,
wherein the external software is arranged in an App of a smart device or in a matched fitting box,
an interface of the external software further comprises at least a retest button, a save button, and a clicking button after hearing.

3. The self-fitting hearing aid having built-in pure tone signal generator according to claim 1,
wherein the external software is connected to the self-fitting hearing aid having built-in pure tone signal generator in a wired or wireless manner for realizing self-fitting.

4. The self-fitting hearing aid having built-in pure tone signal generator according to claim 3,
wherein the pure tone signal generator of the external software is connected to the hearing aid without built-in hearing test in a wired or wireless manner for realizing self-fitting.

5. The self-fitting hearing aid having built-in pure tone signal generator according to claim 1,
wherein the external software is provided with the pure tone signal generator that is used for connecting to the hearing aid without built-in pure tone signal generator for performing self-fitting; the provided pure tone signal generator sends test signals to a user through a mobile phone headset or the hearing aid, thereby forming hearing loss compensation parameters.

6. The self-fitting hearing aid having built-in pure tone signal generator according to claim 5,
wherein the hearing loss compensation parameters comprise one or a combination of frequency, decibel, inflection point, and compression ratio.
